(19) United States
(12) Patent Application Publication
(10) Pub. No.: US 2015/0126929 A1
(43) Pub. Date: May 7, 2015

(54) DERMAL FILLER INJECTOR

(71) Applicant: Allergan, Inc., Irvine, CA (US)

(72) Inventors: Ethan Franklin, Goleta, CA (US); Edwin Kayda, Santa Barbara, CA (US); Zachary Dominguez, Santa Barbara, CA (US)

(21) Appl. No.: 14/533,021

(22) Filed: Nov. 4, 2014

Related U.S. Application Data
(60) Provisional application No. 61/899,768, filed on Nov. 4, 2013.

(51) Int. Cl. A61M 5/20; A61M 5/142; A61M 5/145

(52) U.S. Cl. CPC A61M 5/20; A61M 5/14546; A61M 5/14244; A61M 2005/2086; A61M 2005/2407
USPC 604/155

(57) ABSTRACT

A dermal filler injector is provided which includes a handpiece and a coupling mechanism for operationally coupling the handpiece with a dermal filler cartridge. The handpiece includes a motor for controllably driving a plunger in the cartridge to provide effective and controlled injection of a dermal filler into skin.

This application claims the benefit of U.S. Provisional Patent Application No. 61/899,768, filed Nov. 4, 2013, the entire disclosure of which is incorporated herein by this reference.

The present invention is generally directed to medical syringes and more specifically directed to syringe systems for controlled injection of dermal fillers.

A number of medical and cosmetic applications involve injection of substances into the body.

A conventional medical syringe is a simple piston pump consisting of a plunger that fits tightly in a cylindrical barrel. The plunger is manually pressed to inside cause liquid medicament or fluid to be expelled through a hypodermic needle fitted to a distal end of the barrel. Surprisingly, other than the materials used to make a syringe, conventional disposable syringes are much the same as the very earliest syringe designs.

Aesthetic gels, for example, hydrogel-based dermal fillers are injectable, viscous gels which are used to plump up skin by adding volume to depressions or wrinkled regions of the skin, thereby decreasing the appearance of such wrinkles, depressions, or lack of volume and restoring a more youthful appearance. These compositions are introduced beneath the skin by injection using a syringe and a rather fine gauge needle. Classic syringe designs are far from optimal for the administration of these compositions, especially those that are highly viscous or cohesive. Such gels require relatively high extrusion forces to be expelled through a needle. Moreover, sometimes these gels are used in only trace amounts and must be introduced into skin with very high precision, for example, when used to fill in very minor depressions or fine wrinkles near sensitive structures of the face. This requires expert control over the injection procedure.

Using a traditional syringe, physicians are required to supply possibly significant force, which may reduce the practitioner's ability to control the injection to the degree and with the precision needed for optimal results. Further, traditional syringes typically require the user's hand to be placed a significant distance from the site of the injection in order to operate the plunger, which may also lead to inaccuracy. Some more recently introduced dermal fillers are especially viscous and cohesive therefor amplifying the need a better device.

The present invention provides a dermal filler injection system which overcomes at least some of the problems mentioned above.

### SUMMARY

Accordingly, a dermal filler injector device is provided for facilitating injection of a dermal filler into the skin. The device can be used with a standard syringe. For example, the device can be operationally coupled with a conventional, pre-filled dermal filler syringe to facilitate dermal filler treatment procedures.

Generally, the device comprises a handpiece having a drive mechanism and a coupling mechanism. The handpiece comprises a housing shaped to be comfortably gripped and easily manipulated by a physician. For example, the handpiece may include opposing flanges for supporting a user's fingers during operation of the device such that the handpiece can be gripped in a manner similar to a manner in which a conventional syringe is typically gripped by a physician.

The coupling mechanism is structured to removably engage the handpiece with a syringe, for example, a conventional, pre-filled dermal filler syringe. Such a dermal filler syringe generally includes a cartridge containing a dermal filler gel and a plunger slidable in the cartridge for ejecting the dermal filler through a needle disposed at a distal end of the cartridge. For example, the cartridge may contain about 0.5 mL to about 1.5 mL, for example, about 0.6 mL, about 0.8 mL, about 1.0 mL, about 1.2 mL, or about 1.4 mL of dermal filler product.

In one embodiment, the drive mechanism is structured to be capable of moving the plunger in the cartridge to cause extrusion of dermal filler product in a controlled manner. In another embodiment, the drive mechanism is structured to be capable of moving the cartridge forward and backward relative to the handpiece, while the plunger remains fixed relative to the handpiece, to cause extrusion of the dermal filler product in a controlled manner.

The handpiece may house a motor for driving the drive mechanism, and a battery for powering the motor. The drive mechanism may comprise any suitable mechanism for driving the movement of the syringe in the handpiece. For example, in one embodiment, the drive mechanism comprises a lead screw and a nut slidable along the lead screw. The nut may be engageable with a component of the syringe, and slidable along a fixed lead screw. In one embodiment, the nut is operationally engageable with the plunger of the syringe. In another embodiment, the nut is operationally engageable with the cartridge of the syringe, such that the cartridge is slidable along the lead screw. In another embodiment, the drive mechanism is structured such that when the coupling mechanism is operationally engaged with the syringe, the nut pulls the cartridge forward and backward while the plunger is held in a fixed position relative to the lead screw.

Each and every feature described herein, and each and every combination of two or more of such features, is included within the scope of the present invention provided that the features included in such a combination are not mutually inconsistent.

Other aspects and features of the invention may be more clearly understood and appreciated with reference to the following Detailed Description and appended Drawings.

### BRIEF DESCRIPTION OF THE DRAWINGS

FIGS. 1A, 1B and 1C show, respectively, a side view, a top view and a cross-sectional view of one embodiment of the invention.

FIGS. 2A and 2B show, respectively, a side view and a cross-sectional view of another embodiment of the invention.

FIG. 3A-3C each show a different housing that may be useful as a part of various handpieces of the invention.

FIGS. 4A and 4B show perspective views of a syringe coupling mechanism portion of an embodiment of the invention, which provides a means for rigidly connecting a ball screw of the handpiece with the plunger tip.

FIGS. 5A-5C show cross-sectional views of the coupling mechanism shown in FIGS. 4A and 4B during coupling of the components.

### DETAILED DESCRIPTION

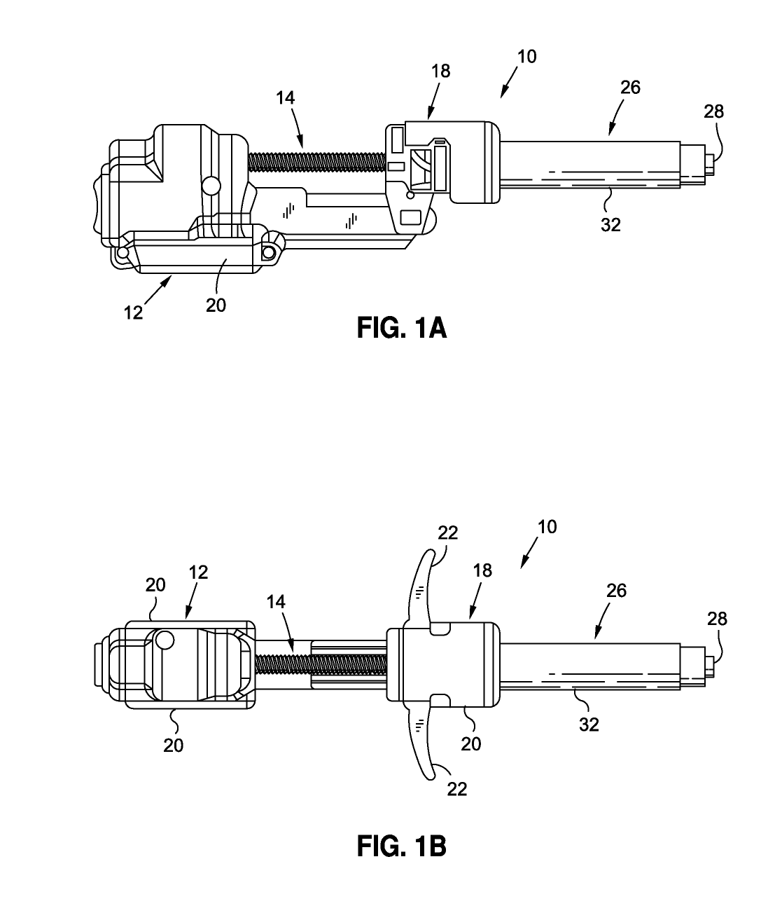

Turning now to FIGS. 1A-1C, various views of a device in accordance with one embodiment of the invention are shown, generally at 10.

The device 10 generally comprises a handpiece 12 having a drive mechanism 14 and a coupling mechanism 18. The handpiece 12 comprises a housing 20. At least a portion of the housing 20 which may be shaped and sized to be comfortably gripped and easily manipulated by a physician. The handpiece 12 may include opposing flanges 22 for supporting a user's fingers during operation of the device 10 such that the handpiece 12 can be gripped in a manner similar to a manner in which a conventional syringe is typically gripped by a physician.

The device 10 may be structured to be couplable to a standard pre-filled dermal filler syringe, or may include a dermal filler syringe 26 as a part of the device 10. The syringe 26 generally includes a hub 28 which is connectable to a needle or cannula (not shown) at a distal end, a barrel/cartridge 32 for containing a dermal filler. Syringe 26 may further include a piston or plunger 34, including a plunger tip 36, movable within the cartridge 32. The cartridge 32 may contain, for example, about 0.5 mL to about 1.5 mL, for example, about 0.6 mL, about 0.8 mL, about 1.0 mL, about 1.2 mL, or about 1.4 mL of dermal filler product, such as a crosslinked, hyaluronic acid based dermal filler product, such as, for example, Juvederm® XC, Juvederm® Voluma, Juvederm® Ultra, or Juvederm® Ultra Plus injectable dermal filler gels, manufactured by and available from Allergan, Inc., Irvine, Calif.

In the embodiment shown in FIGS. 1A-1C, the drive mechanism 14 is configured to move the cartridge 32 of the syringe while holding the plunger 34 or plunger tip 36 in a fixed position with respect to the handpiece 12.

For example, drive mechanism 14 includes a motor 38 for converting electrical power to mechanical power and a gear reduction for speed reduction and force multiplication. The drive mechanism 14 may further comprise a screw and nut assembly, for example, a ball screw assembly 40 comprising a ball screw 42 and ball nut 44 for converting rotational to linear motion, as well as force multiplication. Other components of the drive mechanism may include electrical components, for example, a battery 46, switch(es), and/or wires, etc., for generating power and supplying power to the motor 38. In some embodiments, drive mechanism 14 is structured to be capable of providing significant force, for example, 50 pounds, 100 pounds or more, of force to extrude a dermal filler from the cartridge through a needle having a gauge of 25 G, 27 G, 30 G, 31 G, 32 G or finer.

In one embodiment, the motor 38 comprises a brushed DC motor. However, other motor technologies, including but not limited to, brushless DC motors, stepper motors, piezo-electric motors, etc. may alternatively or additionally be employed within the scope of the invention. These motors may be configured with a single or multistage planetary gearhead that reduced the output speed and increases the available torque.

In one embodiment, the drive mechanism may be a “sliding drive” in which a lead screw is driven (rotated by motor) and a nut is held in a state of anti-rotation. The “sliding drive” functions by causing the nut to travel forward and back along the length of the screw as the screw rotates. The nut may be connected to a plunger which then drives a plunger tip forward in the syringe barrel, causing the dermal filler to be extruded.

In the embodiment show in FIGS. 1A-1D, the drive mechanism 14 is a sliding drive which is configured such that the ball nut 44 moves along the ball screw 42 and pulls the syringe cartridge 32 back and forth, while plunger tip 36 is help in a fixed position relative to the drive mechanism 14 and handpiece 12. Advantageously, in this embodiment, device 10 allows for the overall length of the device 10 to decrease as dermal filler is extruded. This configuration advantageously provides a tactical sensation to the user that, as product is being extruded, the distance between the distal syringe tip and the proximal rear portion of the drive train becomes shorter. This shortening is analogous to the motion or feel a physician traditionally experiences when utilizing a normal, conventional barrel/piston manual syringe.

The device 10 may comprise any further components necessary or desirable for enabling or facilitating operation by a user in accordance with the objectives described elsewhere herein.

For example, in the shown embodiment, the device 10 further includes a counter rotation housing 54, bearing 56, gear 58 attached to ball screw 42, switch 60 to enable user activation of the drive mechanism 14 for delivery of dermal filler and aspiration, motor output gear 62, and thrust bearing 66.

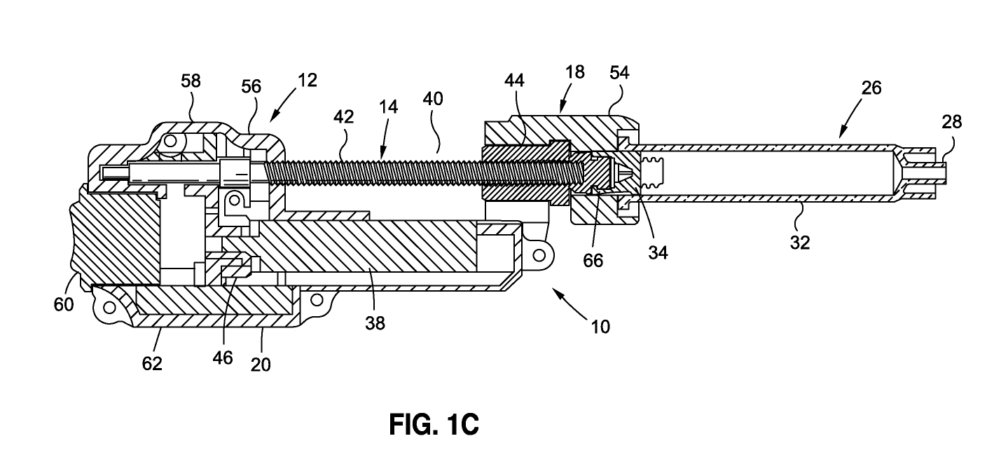

Turning now to FIGS. 2A and 2B, device 110 is provided in accordance with another embodiment of the invention. Device 110 includes handpiece 68 which is operationally couplable to a syringe, for example, a conventional dermal filler syringe. Device 110 is similar to device 10, with some differences. For example, rather than the “sliding drive” described and shown with respect to device 10, device 110 comprises a “side-by-side” drive mechanism 70, and a coupling mechanism 71.

The coupling mechanism is structured to removably engage the handpiece with a syringe, for example, a conventional, pre-filled dermal filler syringe. Such a dermal filler syringe generally includes a cartridge containing a dermal filler gel and a plunger slidable in the cartridge for ejecting the dermal filler through a needle disposed at a distal end of the cartridge. The cartridge may contain about 0.5 mL to about 1.5 mL, for example, about 0.6 mL, about 0.8 mL, about 1.0 mL, about 1.2 mL, or about 1.4 mL of dermal filler product.

In the shown embodiment, the drive mechanism 70 is generally configured such that a nut 72 is driven (e.g. rotated by motor 74 and powered by battery 75) and a lead screw 76 is prevented from rotating.

This may be arranged in any suitable manner. For example, the “side by side” drive mechanism 70 functions by causing screw 76 to move forward relative to the rest of the drivetrain as ball nut 72 rotates about screw 76, but remains fixed with respect to the handpiece. This motion moves a plunger tip 78 located in syringe 26 forward, causing product, e.g. dermal filler, loaded in the syringe cartridge 32 to be extruded. Suitable gears may be provided in order to affect the overall reduction of the system and to transfers power to an offset parallel axis.

Like device 10, device 110 may comprise any further components necessary or desirable for enabling or facilitating operation by a user in accordance with the objectives described elsewhere herein. For example, in the shown embodiment, the device 110 further includes a syringe holder/screw counter rotation housing 80, ball nut 82, gear 84 attached to ball nut 72, bearing 86, motor output gear 88, bearing 90, thrust bearing 92, and switch 70 to enable user activation of the drive mechanism 70.

The handpiece 68 comprises a housing 94 shaped to be comfortably gripped and easily manipulated by a physician. Device 110 further includes ring 98 and flange 99, which may be molded as part of the housing 94, for facilitating handling of the device 110, preventing drops of the device 110, and/or facilitating quick hand-off of device 110 to an assistant, for example.

An encoder or other method of monitoring the number of motor revolutions may optionally be included. An output gear may either drive the lead screw or the nut. In a particular embodiment, a ball screw is provided for this application, providing a high efficiency for use with less robust transmission components, that is, for example, a smaller motor, a smaller battery and electronics. It can be appreciated that a less efficient screw may be used if the motor has sufficient power to overcome the losses.

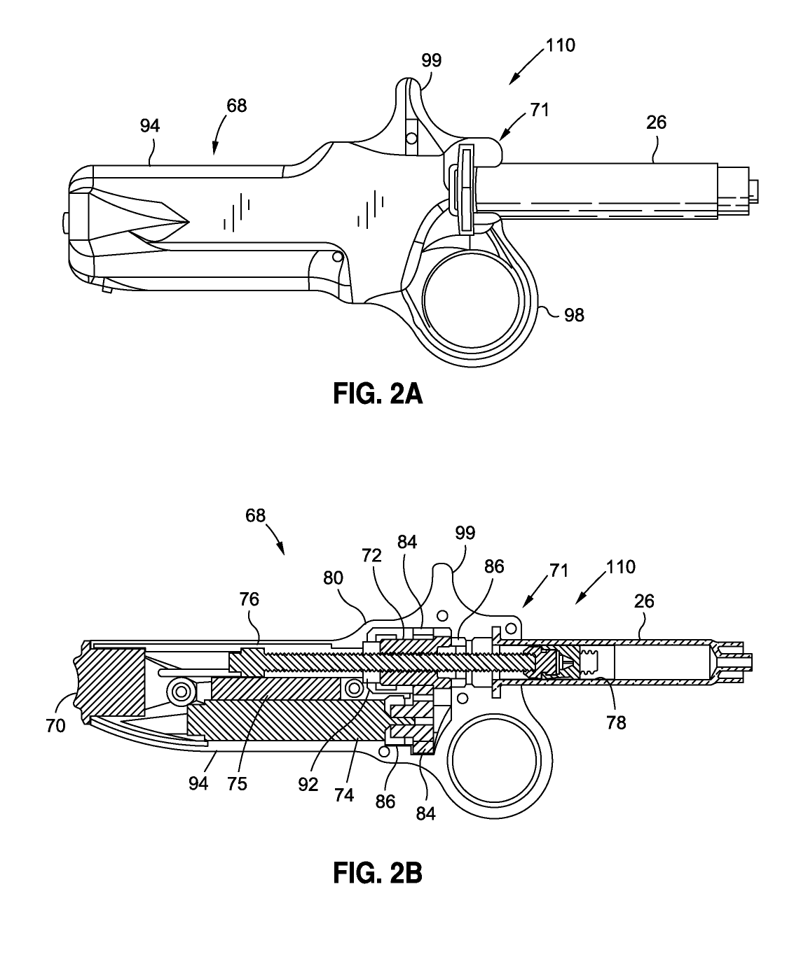

Alternative handpiece housings 116a, 116b and 116c are shown in FIGS. 3A-3C, including various rings 106 and grips 108 for either the thumb location and/or finger location(s). Each of housings 116a, 116b and 116c includes a distal end 120a, 120b, 120c, respectively, structured to be coupled to a syringe (not shown) as described and shown elsewhere herein, and a proximal end 122a, 122b, 122c, respectively, which may include switch 70 as described and shown elsewhere herein. These housings 116a, 116b, 116c, and permutations thereof, may be used as a part of any of the embodiments of the invention described herein, for example, device 10 and 110, with appropriate modification as needed.

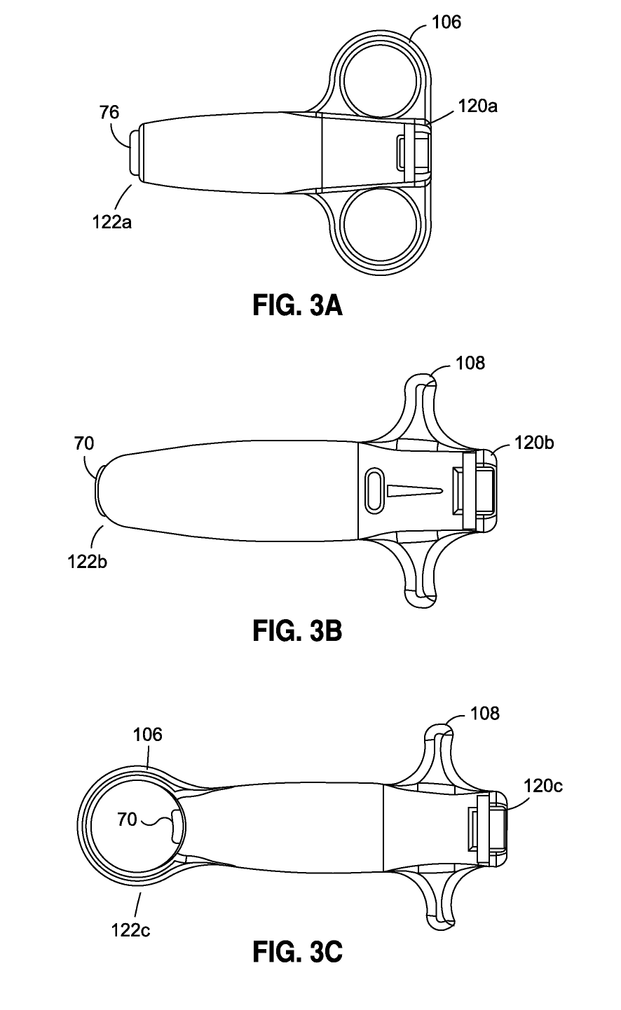

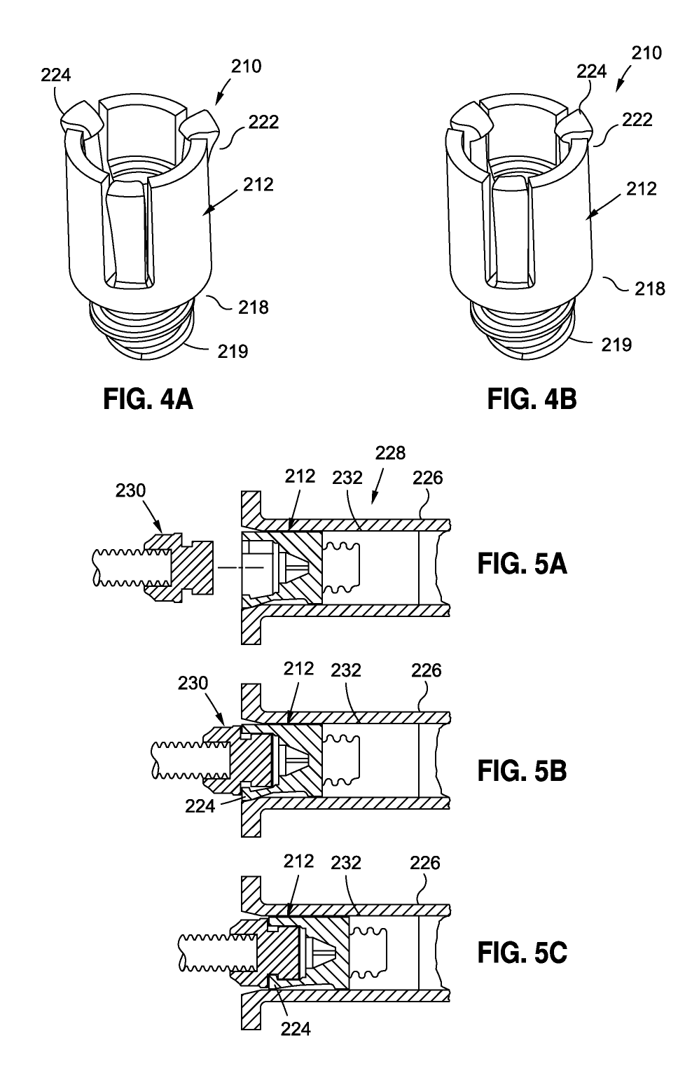

FIGS. 4A and 4B show perspective views of an optional coupler 210 of the invention. FIGS. 5A-5C show cross-sectional views of the coupler 210 shown in FIGS. 4A and 4B.

Optional coupler 210 may comprise clip 212 which provides a means of rigidly connecting a ball screw of the drive mechanism described elsewhere, with a plunger tip in the cartridge. The clip 130 allows the ball screw and plunger to be fixed together in order to facilitate reversing of the plunger in cartridge to achieve aspiration. A front end 218 of clip 212 connects to the plunger tip 232 and both are disposed in syringe cartridge 226. Clip 212 may be connected to plunger tip, for example, screwed into plunger tip 232 using threads 219, or otherwise fixed thereto, or may be integral with plunger tip 232.

A rear portion 222 of the clip 212 features multiple features, for example, flared elements 224, which in their unstressed state flare out slightly (FIG. 4A). The flared elements 224 provide a mating geometry to enable plunger rod 230 to slide into the clip 212. The structure of the clip 212 is such that when the cartridge 226 is full with dermal filler gel, the flared elements 224 are located just outside the cartridge 226 of syringe 228 (FIG. 5A), and therefore allow mating between clip 212 and plunger rod 230 (FIG. 5B). As the device begins to inject, the plunger rod 230 mates with the clip (FIG. 5C) pushing the clip 212 and the plunger tip forward in the cartridge 226. As the clip 212 moves forward the flares 224 get compressed by the inner diameter of the cartridge 226, capturing the geometry of the plunger rod 230. When the flares 226 are compressed, the plunger rod 230 and plunger tip 232 move as a single unit, either forward or backward. There may or may not be a rotational degree of freedom between these two parts. To remove the plunger rod 230 from the clip 212, the plunger rod 230 can be pulled backward out of the syringe (pulling the clip 212 as it moves) until the clip 212 is far enough back such that the flares 224 will revert back to their “open” state.

FIG. 5A shows plunger rod disengaged from clip 212; FIG. 5B shows plunger rod moved forward to drive clip 212 forward down barrel/cartridge of syringe; FIG. 5C shows plunger rod engaged with clip 212, allowing for forward or reverse motion.

While this invention has been described with respect to various specific examples and embodiments, it is to be understood that the invention is not limited thereto and that it can be variously practiced within the scope of the invention.

What is claimed is:

1. A dermal filler injector device comprising:
a syringe comprising a cartridge for containing a dermal filler, and a plunger;
a handpiece having a drive mechanism and a coupling mechanism;
the coupling mechanism structured to removably engage with the syringe; and
the drive mechanism capable of moving the cartridge forward and backward relative to the handpiece, while the plunger remains fixed relative to the handpiece.

2. The device of claim 1 wherein the drive mechanism comprises a motor and a battery.

3. The device of claim 1 wherein the drive mechanism comprises a lead screw and a nut.

4. The device of claim 3 wherein the coupling mechanism comprises a nut engageable with the cartridge and slidable along the lead screw.

5. The device of claim 3 wherein the drive mechanism is structured such that when the coupling mechanism is engaged with the syringe, the a nut pulls the cartridge while the plunger is held in a fixed position relative to the lead screw.

6. The device of claim 1 wherein the handpiece includes flanges for supporting a user's fingers during operation of the device.
